(19) United States
(12) Patent Application Publication
(10) Pub. No.: US 2009/0264720 A1
(43) Pub. Date: Oct. 22, 2009

(54) Wearable Automated Blood Sampling and Monitoring System

(75) Inventors: Marc C. Torjman, Southampton, PA (US); Michael E. Goldberg, Philadelphia, PA (US)

Correspondence Address: COZEN O'CONNOR, P.C., 1900 MARKET STREET, PHILADELPHIA, PA, 19103-3508, US

(73) Assignee: The Cooper Health System, Camden, NJ (US)

(21) Appl. No.: 12/104,503

(22) Filed: Apr. 17, 2008

(51) Int. Cl. A61B 5/157; A61B 5/05; A61B 5/1468

(52) U.S. Cl. 600/322; 600/345; 600/347; 600/361

(57) ABSTRACT

A wearable blood chemistry monitoring device is disclosed which comprises a wearable automated blood chemistry monitoring device comprising (A) a mini pump which can be, for example a peristaltic pump or syringe pump; (B) a portable form factor mechanical apparatus which preferably includes a rotatable disc with a hole which fits over the pump; (C) at least one measurement element for measuring at least one blood parameter, preferably on the disc, and preferably a series of glucose strips arranged radially in a spoke-like pattern on the disc; (D) a catheter connected to the pump via a tube; (E) a computerized device adapted to automatically measure blood analytes and blood parameters; (F) a belt adapted to hold the housing, a waste bag, and a flush solution bag; wherein the pump and disk are arranged in the housing so that a hole in a disk fits over the pump.

### BACKGROUND OF THE INVENTION

This invention relates to blood chemistry monitoring systems and more particularly to wearable blood sampling and monitoring, especially for glucose monitoring.

Various systems and methods are known and used for measuring and analyzing blood drawn from patients. Usually blood is drawn and sent to a laboratory for analysis, and then the results are reported to a medical professional who then decides among treatment options.

In certain hospital situations, for example when administering insulin therapy, frequent blood measurements are needed. In the case of insulin therapy, it is glucose measurements which are needed. In other cases, frequent hematology data, lactate measurements, or other analytical measurements are needed rather than, or in addition to, glucose measurements to aid in managing a patient's condition.

Using current systems and methods, clinical laboratory measurements of blood drawn from patients only provide sporadic data which are insufficient to guide certain therapies where patients would benefit from optimal drug titrations to maintain certain physiologic parameters within clinically optimal ranges.

For example, glucose control and insulin drug delivery for hyperglycemia could benefit from continuous or semi-continuous, automated blood sampling and analysis in order to automatically regulate insulin delivery. This approach is also applicable to other analytes and drug therapies such as with anticoagulant administration. The advantages of automated blood sampling in the cases of insulin therapy for example have been recognized and various systems have been proposed to achieve such sampling and analysis.

For example, Wong, in U.S. Pat. Nos. 5,165,406; 5,758,643; and 5,947,911. discloses a system for monitoring a patient's blood chemistry which intermittently draws blood samples into a special sensor assembly having a plurality of analytical sensors, each sensitive to a particular blood parameter. A catheter connects the sensor assembly to the patient.

Goldberger, et al., in U.S. Pat. Pub. 20080014601, disclose a glucose measurement system which has a controller, a pump, and flush solution in a first reservoir, IV solution in a second reservoir, a first valve, a second valve, and a plurality of tubing placing the pump, reservoirs, and valves in fluid communication with each other. The pump is a syringe pump.

Goldberger, et al., in U.S. Pat. Pub. 20070123801, disclose a wearable, automated blood testing device in an inflatable cuff which employs a lancet, a lancet launching mechanism, and a blood analyte measuring element, and a control unit for controlling the periodic sampling of blood and measurement of blood analytes and blood parameters, wherein the control unit is programmable to initiate blood sampling for measurement of blood analytes at pre-determined time intervals. Capillary forces are used to carry the blood to a reservoir where the blood sample is carried through small passages to a blood analyte measuring element contained within a cartridge, or the blood analyte measuring element may be integrated with the lancet.

For many situations it is undesirable to have an automated lancet needle system due to the cost and difficulty of engineering a system which involves multiple needles. Furthermore, it is believed that the use of multiple needles is impracticable due to cost and risk of failures in operation.

It is therefore an object of the present invention to provide a portable, wearable system for enabling frequent and automated blood sampling in a patient without use of lancets or automated needle punctures in the patient. It is another object to provide automated blood analysis employing a catheter which remains in place and involves only one puncture rather than the multitude of punctures which a lancet system requires. Also, smaller diameter needles are known to be prone to acclusion, therefore, reducing the reliability of such systems.

### SUMMARY OF THE INVENTION

These objects, and others which will become apparent from the following detailed description and drawings, are achieved by the present invention which comprises in one aspect a wearable blood chemistry monitoring device comprising (A) a mini pump, (B) a portable form factor mechanical apparatus; (C) at least one measurement element for measuring at least one blood parameter (D) a catheter connected to the pump via a tube, (E) a computerized device adapted to automatically measure blood analytes and blood parameters (F) a belt adapted to hold a housing, a waste bag, and a flush solution bag; wherein the pump and a disk are arranged in the housing so that a hole in the disk fits over the pump.

The device of the invention is for use in patients with central or peripheral access catheter ports and functions to automatically monitor blood sampling and measurement of various blood analytes such as glucose, electrolytes, lactate, hemoglobin, and hematocrit, as well as any agent, drug or blood test, which may be quantitated with microliter blood quantities on a strip, cuvette or other body fluid type assay.

The blood monitoring device of the invention consists of regular sterile tubing but preferably consists of microtubing. The tubing is connected to a mini pump (i.e., peristaltic) with rotating valves or other flow control systems which allow bidirectional flow for drawing blood and then flushing the tubing in the reverse mode.

Preferably the monitoring device comprises an optical cuvette type window for optical measurement of blood. The device further includes a rotatable disk located within a circular housing which is adapted to fit in a belt or other wearable apparatus such as an armband, leg band, or waistband. The rotatable disk has a hole in the center and carries a series of glucose test strips, preferably in a radial, i.e., spoke like configuration. Other configurations of test strips may be used in some embodiments. The glucose strips are preferably read using optical or other reading systems. In the glucose monitoring embodiments, the data can be used to control a closed loop insulin delivery system, and/or to initiate an alarm, and the data can be transmitted by various means to any type monitor, either locally or remotely. Wireless transmission of blood data can be utilized. Alternatively, the blood data may be monitored locally, on the device itself.

In a closed loop insulin delivery system wherein glucose is being periodically tested, a controller and an algorithm can be employed to use blood glucose data from the monitoring device to deliver insulin via intravenous or subcutaneous routes when appropriate.

The monitor of the invention can be used by critically ill or perioperative, i.e., around the time of surgery, patients needing close monitoring of medical conditions requiring single or multiple laboratory parameters to guide therapy and monitor patients conditions and progress.

The monitor of the invention can provide patients and medical staff advantages in not having the patient tethered to a monitor which is typically placed on a pole or at the patient's bedside.

In the case of sedated or anesthetized patients, frequent readings from the monitor on anesthetic drug concentrations would permit the delivery of anesthesia care using more optimal drug concentrations and the delivery of anesthesia care using optimized drug concentrations and the delivery of care using closed loop feedback with input from other monitors for information on physiologic parameters.

In a closed or semi-closed loop system for automated delivery of anesthetics, the monitor would provide real time or semi-real time blood chemical data while other physiologic parameters would be received by the anesthetic delivery system from other sources. In such embodiments, blood chemistry in addition to glucose is monitored.

The monitor can be used in certain embodiments on an ambulatory patient who has available long term central access ports. The monitor can also be used with peripheral catheters in a hospitalized patient or an ambulatory patient since only small blood volumes are used for sampling and analysis by the monitor.

While it is preferred that the housing be circular to accommodate the rotatable disk, the housing can be other portable form factor as long as the disk and the mini pump are accommodated.

The pumping action for the drawing of the blood and flushing of the fluid lines and catheters is provided by the mini peristaltic pump.

The monitor is designed to permit using a multianalyte disc with strips mounted at intervals around the disc, for example in a radial pattern at regular intervals, spaced evenly around the disc. The strips are advanced by controlled rotation of the disc so that they are at the proper position and aligned with the blood dispensing orifice.

The monitor in most embodiments requires two small reservoir bags, one containing the flush solution such as saline or other injectable solution for purging the tubing, and the other for receiving waste solution from the purging. In other embodiments no waste bag is necessary and the physiologic fluid solution is flushed back to the patient in a closed system.

The invention shall be described in greater depth in the drawings and detailed description provided herein.

### BRIEF DESCRIPTION OF THE DRAWINGS

These and other features and advantages of the present invention will be illustrated by certain embodiments set forth in the detailed description when considered in connection with the accompanying drawings, wherein:

FIG. 1 depicts a patient wearing a blood chemistry monitoring device with catheter and tubing.

FIG. 2 depicts a schematic view of a blood chemistry monitoring device including catheter tubing and a flush solution bag.

FIG. 3 is a schematic view of a blood chemistry monitoring device according to the invention with a mini peristaltic pump arranged in the center.

FIG. 4 is a blood chemistry monitoring device according to the invention with a waste bag and flush solution bag, a mini peristaltic pump, and a central catheter depicted.

FIG. 5 is a blood chemistry monitoring device according to the invention with a waste bag and flush solution bag, a mini peristaltic pump, and a central catheter depicted.

FIG. 6 shows a typical patient wearing a blood chemistry monitoring device according to the invention with a waste bag and flush solution bag, a mini peristaltic pump, and a central catheter depicted.

FIG. 7 shows a typical patient wearing a blood chemistry monitoring device with one bag according to the invention.

FIG. 8 shows a block diagram of the Device Controller.

### DETAILED DESCRIPTION

The present invention provides a wearable, automated system for sampling and monitoring of blood analytes and blood parameters. The system components are combined in a single apparatus to initiate automatic, periodic blood sampling and monitoring. The system operates automatically to draw blood samples at suitable, programmable frequencies to analyze the drawn blood samples and obtain the desired blood readings such as glucose levels, hematocrit levels, hemoglobin blood oxygen saturation, blood gasses, lactates or any other parameter as would be evident to persons of ordinary skill in the art.

The system includes a reusable sensor for obtaining blood measurements. The sensor is preferably electrochemical or optochemical sensor, but other options such as sensors that support optical blood measurements (without relying on chemical reactions between the sample of blood and a chemical agent embedded in the sensor) are disclosed. The present invention also discloses apparatuses and methods that employ components of manual test systems (e.g. blood glucose test strips) for use in an automated measurement system.

As referred to herein, the terms “blood analyte(s)” and “blood parameter(s)” refers to such measurements as, but not limited to, glucose level; ketone level; hemoglobin level; hematocrit level; lactate level; electrolyte level (Na.sup.+, K.sup.+, Cl.sup.−, Mg.sup.2+, Ca.sup.2+); blood gases (pO.sub.2, pCO.sub.2, pH); blood pressure; cholesterol; bilirubin level; and various other parameters that can be measured from blood or plasma samples.

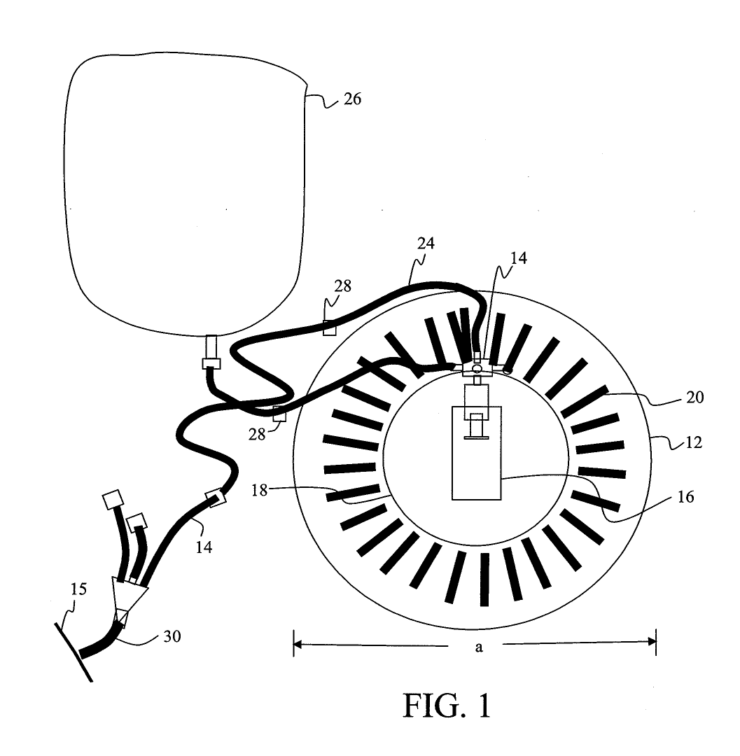

Referring now to FIG. 1, an embodiment of a blood chemistry monitoring device 11 according to the invention is shown having a portable form factor i.e., a circular housing 12 (around 10 cm or 3.94 inches in some embodiments), safety valves 28, 28, port 14, mini pump 16, rotatable disk 18, blood chemistry test strips 20, central catheter 30, flush solution bag 26, electronic meter 23, device controller 31. Device 11 is worn by a patient 15 by means of a belt 17 adapted to hold device 11. Device 11 receives blood from patient 15 through a tube 29 in fluid connection to a central catheter 30 placed in a vein of patient 21. Device controller 31 operates safety valve 28 and mini syringe pump 16, which is located in central cavity 19 of rotatable disk 18 allowing blood to flow from patient 15. Blood chemistry test strips 20 are arranged radially from the central cavity toward the circumference of rotatable disk 18 in a star pattern. A mechanism 32 is provided to advance disk 18 in direction 27 by the distance between each glucose test strip 20. One test strip 20 at a time is moved into position by means of mechanism 32, controlled by device controller 31 to receive a drop of blood from a port 33. The device includes a flush solution bag 26 in fluid connection with the device via tubes 29 and 24, respectively. After blood is received via tube 22 from patient 15, then tube 22 is flushed with solution from bag 26. Device 11 also returns the solution to the patient via the same tube 29 when the pump 16 is reversed and safety valve 28 is operated by device controller 31. Safety valves 28 also prevent reflux back to the patient.

Glucose test strips 20 are read after they have received a drop of blood and have time to react chemically, depending on which types of blood chemistry are to be determined and the result is displayed on electronic meter 23 over hard-wired link 37 or wireless communication methods, as are well-known in the art.

Housing 12 is not necessarily round but can be any desired shape, usually a portable form factor designed to fit into belt 17. Belt 17 can alternatively be a waste band, arm band, leg band, or any other apparatus which may be worn by patient 15.

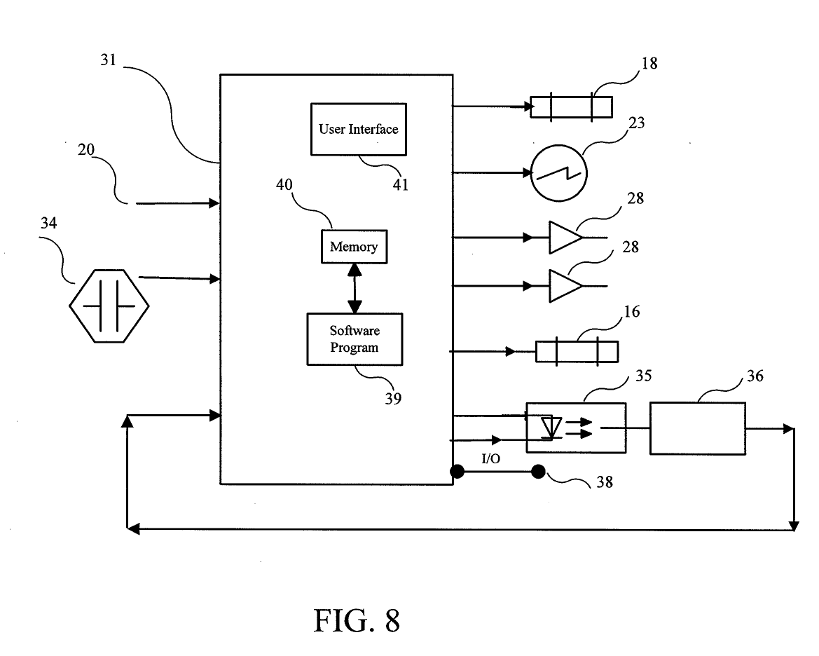

Referring to FIG. 8, the diagram depicts the components of a device controller as used in the automated blood parameter sampling and monitoring system of the present invention. Device controller 31 preferably comprises software program 39, memory 40 and user interface 41. Device controller interfaces to the monitoring device via user interface 41 and I/O ports. Fluid sensor 34, blood chemistry test strip 20 and output of light detector 36 are all connected to the input of the controller 31. Both safety valves 28, electronic meter 23, rotatable disk 18, mini pump 16 and light source 35 are under the control of the device controller.

Software program 39 is used for data analysis and correlation. Additionally, software program 39 also supports calculation of trends using look-up tables and algorithms based on measurement history. The results of data analysis and interpretation performed upon the stored patient data by the monitor may optionally be displayed in the form of a paper report generated through a printer (not shown), besides being displayed on the electronic monitor screen 23. Software 39 uses a blend of symbolic and numerical methods to analyze the data, detect clinical implications contained in the data and present the pertinent information in the form of a graphics-based data interpretation report. The symbolic methods used by the software to encode the logical methodology used by expert diabetologists as they examine patient logs for clinically significant findings, while the numeric or statistical methods test the patient data for evidence to support a hypothesis posited by the symbolic methods which may be of assistance to a reviewing physician.

Device controller 31 is also preferably equipped with an I/O port 38 that may optionally include interfaces to external automated systems such as, but not limited to, portable monitors, printers, hospital data network(s), external processors and display units, and other monitoring automated systems. The connection between the device controller and the various possible external units can be made via any of the known wired or wireless communication methods, as are well-known in the art. Alternatively, I/O port 38 may be adapted to provide telemetry.

Software program 39 allows the user to perform queries on the stored information. For example, the user may wish to view the results of previous measurements or the current measurement. The user may set an alarm, when the sensor is in operation, or reconfigure the port assigned to a component. The automated system further includes alerts and integrated test systems. The alerts may include alerts for hyperglycemia and hypoglycemia. The alerts may also include alerts for hemoglobin level below a defined level. The device alerts when the blood measurement falls outside a defined range for blood parameters.

Software program 39 uses a blend of symbolic and numerical methods to analyze the data, detect clinical implications contained in the data and display the pertinent information. The symbolic methods used by the software encode the logical methodology used by expert diabetologists as they examine patient logs for clinically significant findings, while the numeric or statistical methods test the patient data for evidence to support a hypothesis posited by the symbolic methods which may be of assistance to a reviewing physician.

The processed data may be transmitted from the monitoring device to a central monitoring station when the automatic blood parameter testing device is used in a hospital environment. The monitoring device maintains a record of all physiological parameters measured over a period of time from different patients. Thus, the monitoring device can communicate with a designated central monitoring station to supply data (telemetry) from previous patients or the current patient.

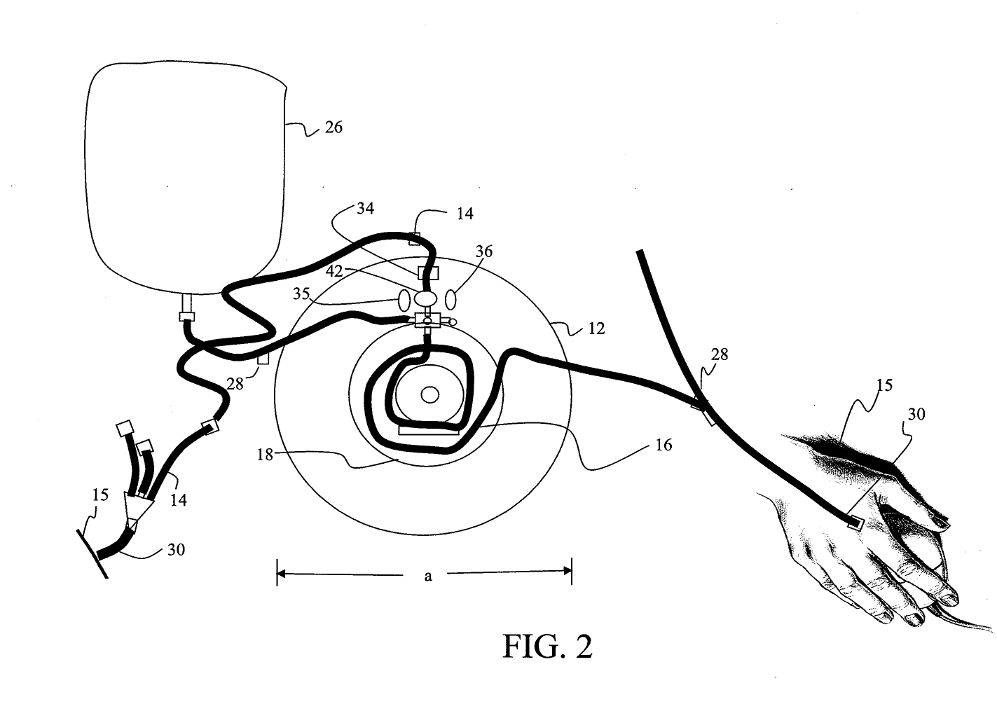

Referring now to FIG. 2, another embodiment of the invention is shown as the same configuration as the embodiment of FIG. 1, except blood chemistry test strips 20 are replaced with a fluid sensor 34, a cuvette of flow thru cell 42, a light source 35, a light detector 36 and mini pump 16 is preferably a peristaltic pump. Cuvette or flow thru cell 42 is preferably a surface or miniature container, such as but not limited to a capillary tube, enabling storage of the blood sample for optical measurements. In this embodiment, both a light source and a light detector are used for measuring the blood analyte based on reflected, transmitted or other known optical effects such as Raman Spectroscopy, NIR or IR Spectroscopy, FTIR or fluoroscopy. Light source 35 and light detector 36 are chosen such that glucose (1650 nm in the Infra Red region of the spectrum) and hemoglobin (540 nm) can be accurately measured and monitored. The operation of the light source and detector is well known in the art.

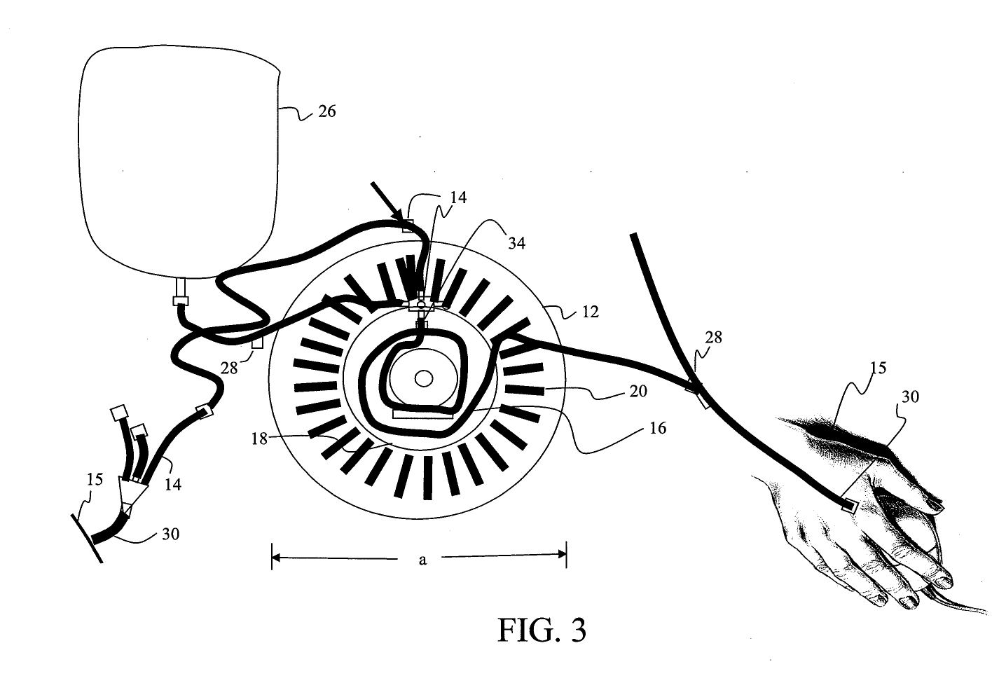

Referring to FIG. 3, in another embodiment, monitor device 11 is shown connected to an IV, which is another option if no central venous catheter (CVC) is available. The device would be connected to the proximal infusion port in the preferred embodiment to avoid sample contamination from the mid and distal infusion ports. The sample draw rate could be adjusted (slow draw, intermittent draw steps with a 1 sec pause between steps) to prevent sample contamination from other infusions.

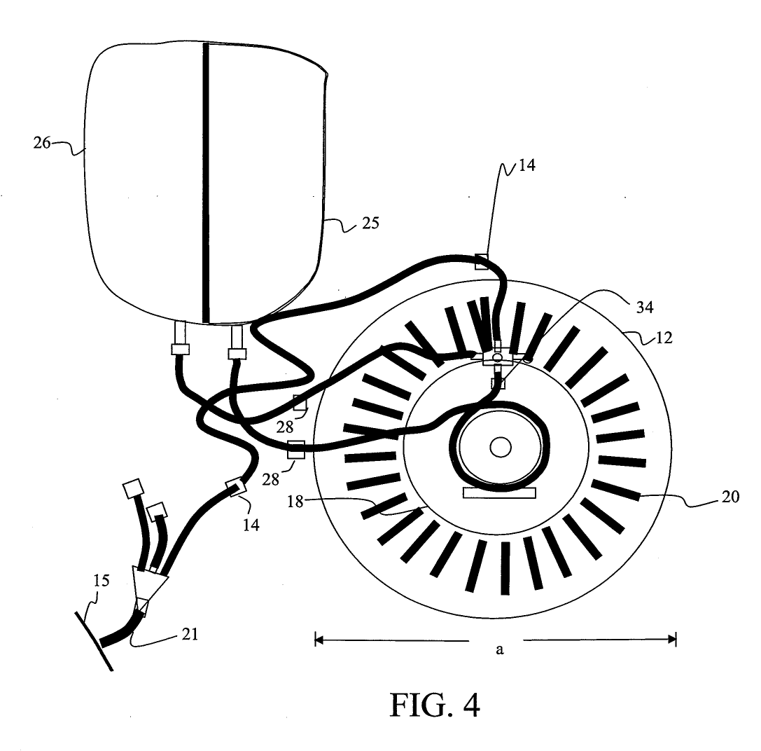

Referring to FIG. 4, in another embodiment, flush solution bag 26 is replaced with a dual compartment bag. One compartment contains flush solution and the other compartment provides for waste storage.

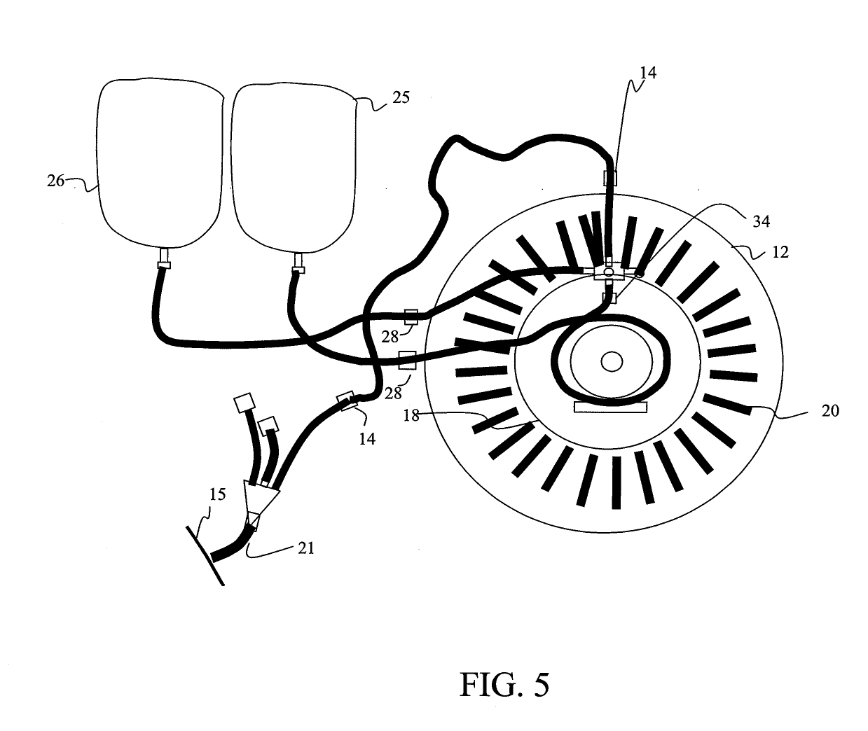

Referring to FIG. 5, in yet another embodiment, flush solution bag 26 is replaced with two distinct bags. One bag contains flush solution and the other provides for waste storage.

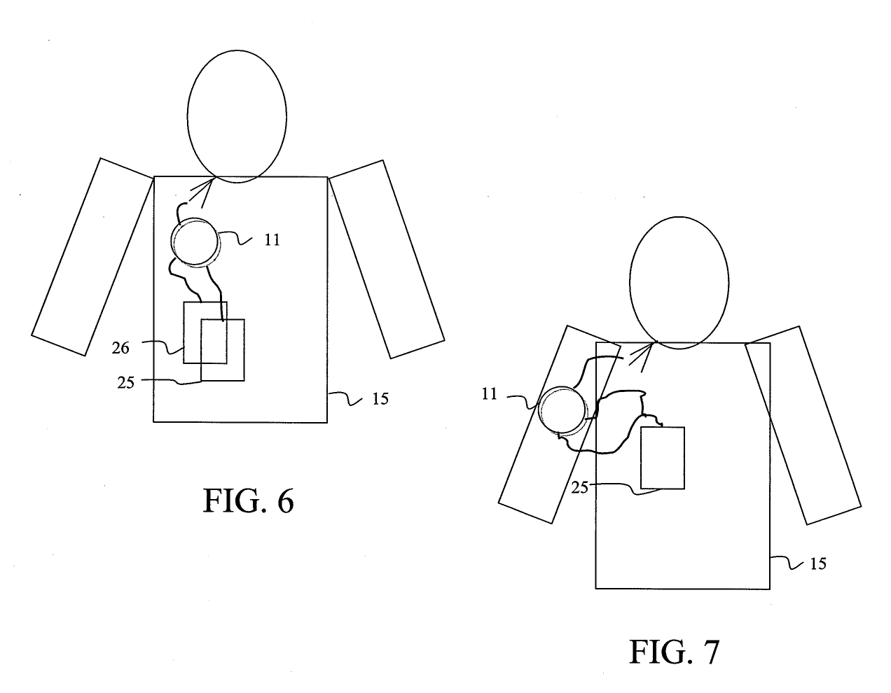

Referring to FIGS. 6 and 7, typical area of the body where the device can be worn is shown.

While the invention has been described in conjunction with specific embodiments, it is not intended to limit the invention to one embodiment. Thus, the present invention is not intended to be limited to the embodiments described, but is to be accorded the broadest scope consistent with the disclosure set forth herein.

What is claimed is:

1. A wearable automated blood chemistry monitoring device comprising (A) a mini pump, (B) a portable form factor mechanical apparatus; (C) at least one measurement element for measuring at least one blood parameter (D) a catheter connected to the pump via a tube, (E) a computerized device adapted to automatically measure blood analytes and blood parameters (F) a belt adapted to hold the housing, a waste bag, and a flush solution bag.

2. The automated device of claim 1, wherein element (B) further comprises a circular housing adapted to fit in a belt adapted to be worn by a patient, the housing including a valve and a port.

3. The automated device of claim 2, further comprising a rotatable disk located within the housing, the disk having a hole in the center, the disk carrying a series of blood chemistry test strips wherein the mini pump is at least one of syringe or peristaltic and the rotatable disk are arranged in the housing so that the hole in the rotatable disk fits over the pump.

4. The wearable automated blood chemistry monitoring device of claim 3 wherein the strips are glucose strips.

5. The wearable automated blood chemistry monitoring device of claim 4 wherein the blood chemistry strips are adapted to measure one or more blood factors selected from the group consisting of glucose, lactate, pH, electrolytes, and drugs.

6. The wearable automated blood chemistry monitoring device of claim 3 wherein the strips are arranged radially.

7. The wearable automated blood chemistry monitoring device of claim 3 further comprising an advancement means to rotate the disk and advance each test strip sequentially and position the strip for direct contact with a blood sample.

8. The wearable automated blood chemistry monitoring device of claim 1 wherein the pump is adapted to dispense 1 to 3 ml into a syringe or well reservoir.

9. The wearable automated blood chemistry monitoring device of claim 1, wherein element (C) further comprises a light source, a light detector and a cuvette or flow thru cell

10. The wearable automated blood chemistry monitoring device of claim 9, wherein the light source, the light detector are used for measuring the blood analyte based on reflected, transmitted or other known optical effects such as Raman Spectroscopy, NIR or IR Spectroscopy, FTIR or fluoroscopy.

11. The wearable automated blood chemistry monitoring device of claim 1 wherein the computerized device is a device controller.

12. The wearable automated blood chemistry monitoring device of claim 1 wherein the device controller is capable of providing telemetry.

13. The wearable automated blood chemistry monitoring device of claim 1 wherein the computerized device further comprises an electronic meter.

14. The wearable automated blood chemistry monitoring device of claim 1 wherein the device is capable of being worn on a patient's arm.

15. The wearable automated blood chemistry monitoring device of claim 1 wherein the device is adapted to fit in a belt to be worn by the patient.

16. The wearable automated blood chemistry monitoring device of claim 1 further comprising a fluid sensor.

17. The wearable automated blood chemistry monitoring device of claim 16 wherein the fluid sensor is an electrochemical sensor capable of detecting the presence of and enabling the measurement of the level of an analyte in a blood sample via electrochemical oxidation and reduction reactions at the sensor.

18. The wearable automated blood chemistry monitoring device of claim 16 wherein the sensor is an optochemical sensor capable of detecting the presence of and enabling the measurement of the level of an analyte in a blood or plasma.

19. The wearable automated blood chemistry monitoring device of claim 1 wherein the device controller determines the oxygenation level of the blood and uses the oxygenation level to calibrate the glucose calculation, determines at least one of a hemoglobin concentration a hematocrit of the blood and calibrates the glucose calculation.

20. A method for automatically measuring blood analytes and blood parameters, the method comprising:
programming a device controller unit to obtain a blood sample at predetermined time intervals wherein a portable form factor mechanical apparatus is activated;
attaching a central catheter to a proximal port of the device;
initiating a blood sample at said predetermined time interval;
measuring analytes and parameters of said blood sample using an analyte measuring element in said blood sampling and measurement unit; and
displaying measurement of said analytes and parameters.
